(19) United States
(12) Patent Application Publication
(10) Pub. No.: US 2002/0055759 A1
(43) Pub. Date: May 9, 2002

(54) Bioactive surgical suture

(76) Inventor: Terry Y. Shibuya, Irvine, CA (US)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 10/012,906

(22) Filed: Nov. 6, 2001

Related U.S. Application Data
(60) Provisional application No. 60/246,543, filed on Nov. 6, 2000.

(51) Int. Cl. A61B 17/04

(52) U.S. Cl. 606/231; 427/2.31

(57) ABSTRACT

The application concerns surgical suture coated with one or more biologically active compounds. The application discloses methods of making coated suture, placing the coated suture in an organism, and methods of using the coated suture in the treatment of diseases such as cancer.

### RELATED APPLICATION

The present application claims the benefit under 35 U.S.C. §119(e) of U.S. Provisional Application No. 60/246,543, filed Nov. 6, 2000.

### BACKGROUND OF THE INVENTION

1. Field of the Invention

The present invention relates to surgical suture coated with one or more biologically active compounds. More particularly the present invention relates to methods of producing biologically active suture, placing biologically active suture in a patient and using biologically active suture in the treatment of disease.

2. Background

The induction of a T-cell immune response by antigen-presenting cells (APCs) occurs in three distinct stages. In the first stage, a nonspecific adhesion occurs when APCs and T-cells randomly interact in the circulation and in lymphoid tissues. Cell surface ligands and adhesion receptors mediate the adhesion. Adhesion is followed by recognition when the antigen-major histocampatibility complex of the APC crosslinks with the T-cell receptor (TcR). Thus the APCs must first process, transport, and present a sufficient quantity of a specific antigen. Endogenous peptides (derived from intracellular proteins) are generally presented with MHC class I (HLA-A, B, or C), whereas exogenously processed peptide antigens (derived from circulating proteins) are generally presented with MHC class II (HLA-DR, DP, or DQ). The final stage occurs when a second or costimulatory signal is provided by the APC to the T-cell, enhancing activation.

The best-characterized second signal for T-cell activation occurs via interaction between the B7.1 or B7.2 ligand of the APCs and CD28 of the T-cell. CD28 is a 44 kDa subunit found on the surface of most CD4⁺ cells and 70% of CD8⁺ cells. The immune response initiated may be characterized as a type 1 (T_{H}1) or 2 (T_{H}2) response based on the cytokines secreted from the CD4⁺ cells. A T_{H}1 response is characterized by the secretion of inter-leukin 2 (IL-2), tumor necrosis factor α (TNFα) and interferon γ (IFN-γ) and represents a cytotoxic response against cancer or microbes. A T_{H}2 type response is characterized by the secretion of IL-4, IL-5, IL-6, IL-10 and IL-13 and enhances antibody production from B cells as well as an allergic response. Anti-CD28 (αCD28 or 9.3) monoclonal antibodies (MoAbs) can substitute for the B7 ligands to stimulate CD28 and to provide a strong stimulatory signal to T-cells (Hara et al. J. Exp. Med. 161:513-1524 (1985)). Costimulation also lowers the concentration of anti-CD3 (αCD3) required to induce a proliferative response in culture.

The generation of cytotoxic T lymphocytes (CTLs) by αCD3/αCD28 costimulation has been established (Azuma et al. Curr. Top. Microbiol. Immunol. 198:59-74 (1995)). One of the advantages of costimulation with anti-CD3/anti-CD28 has been resistance to immunosuppression, such as that seen in patients with head and neck squamous cell carcinoma (HNSCC). A “hierarchy of immunosuppression” exists in HNSCC patients. Immune reactivity as measured by proliferation, cytotoxicity, and natural killer (NK) cell activity is maximally suppressed in tumor infiltrating lymphocytes (TIL), followed by proximal lymph node lymphocytes (LNL), distal LNL and peripheral blood lymphocytes (PBL) (Wang et al. Otolaryngol. Head Neck Surg. 105:517-527 (1991)). The function of NK cells from regional lymph nodes is also inhibited. TIL, LNL, and PBL activity is significantly less in HNSCC patients than controls. In particular, IL-2 induced cytotoxicity is suppressed in LNLs proximal to the tumor compared to LNLs distal to the tumor.

Co-activation of resting T-cells with αCD3/αCD28 has enhanced proliferation, cytokine production, and cytotoxicity. αCD3/αCD28 costimulation enhances expression of IL-1α, IL-2, IFN-γ, GM-CSF, lymphotoxin, and chemokines. Several of these cytokines have demonstrated benefit in the treatment of HNSCC. Costimulation also enhances the production of lymphokines by CD4⁺ cells through transcriptional and post-transcriptional regulation of gene expression.

The cytolytic activity of αCD3/αCD28 activated cells is from small resting T-cells and CD4⁺ cells. The lymph nodes of HNSCC patients are filled with small resting T-cells and CD4⁺ cells that have tremendous potential for transformation into anti-tumor cytotoxic T-lymphocytes. Thus patients with HNSCC are prime candidates for an effective, targeted treatment with αCD3 and αCD28.

Anti-viral effects of αCD3/αCD28 have also been identified. CD4⁺ T-cells from HIV+ patients have been activated and expanded ex vivo using αCD3/αCD28 coated beads. HIV-1 viral load declined in the expanded T-cell population in the absence of any anti-retroviral agents. Additionally, αCD3/αCD28 stimulated CD4⁺ T-cells, from uninfected donors, appear to be resistant to HIV-1 infection.

The therapeutic efficacy of T-cells activated by costimulation with αCD3/αCD28 is being evaluated in patients with melanoma, lymphoma and various solid tumors (Renner et al. Science 264:833-835 (1994)). For example, T-cells have been stimulated ex vivo with αCD3/αCD28 immobilized on magnetic beads or tissue culture plastic and used in adoptive immunotherapy trials (Levine et al. Science 272:1939-1943 (1996); Thompson et al. Proc. Natl. Acad. Sci. U.S.A. 86:1333-1337 (1989)). Immobilized αCD3/αCD28 is used because it has been found that αCD3 and αCD28 have a stronger immunologic effect when they are immobilized. However, there are several problems with αCD3/αCD28 immobilized on beads or plastic. For example, if stimulation is done ex vivo, there are risks of contamination and infection. If the stimulation is done in vivo, it is difficult to remove the beads if an undesired reaction occurs. Because the beads are small, it is not feasible to remove them individually. Removal thus requires an invasive surgical procedure and often requires the removal of tissue along with the beads.

Surgical suture is ordinarily composed of an inert substance, and was created for the purpose of closing wounds. It is commonly used to facilitate wound healing by the approximation of tissues. Suture may be resorbable or permanent in nature depending upon the type of material from which it is made. Suture is typically designed to cause minimal tissue reaction, so wound healing is not impaired by its presence.

### SUMMARY OF THE INVENTION

One aspect of the present invention is a method of coating surgical suture with one or more biologically active compounds. A length of surgical suture is placed into a solution comprising the biologically active compounds and the suture is incubated in the solution until the biologically active compounds are bound to the suture.

The suture used may be selected from the group consisting of polyester suture, vicryl suture, nylon suture, plain gut suture, polyglactin suture, chromic gut suture and silk suture.

The solution in which the suture is incubated may be phosphate buffered saline. In another embodiment the phosphate buffered saline is Ca²⁺ and Mg²⁺ free.

The incubation may be carried out for 18 hours. In another embodiment the incubation is carried out at 37° C.

In one embodiment the biologically active compounds are present in the solution at a concentration of 2 μg/ml.

At least one of the biologically active compounds may be a monoclonal antibody. In one embodiment the biologically active compounds are αCD3 and αCD28.

Another aspect of the present invention is a method of placing a surgical suture coated with one or more biologically active compounds into a patient. A needle with a trochar is passed into the desired location in the patient. The trochar is removed and the suture is passed down the barrel of the needle. The needle is then removed over the suture, leaving the suture in the desired position.

In one embodiment the position of the needle is determined prior to its removal from the patient. The position of the needle may be determined by manual palpation, ultrasound, CT scan or radiograph. In another embodiment the suture itself is labeled such that its location can be determined by radiography.

In yet another embodiment the proximal end of the suture is secured to the skin of the patient.

Another aspect of the invention is a method of reducing rejection of transplanted organs. A suture coated with one or more immunosuppressive compounds is placed in the organ donor bed or the regional lymphatics of a patient. The suture may be coated with an antibody. In one embodiment the antibody is αCD3.

### BRIEF DESCRIPTION OF THE DRAWINGS

FIG. 1 A, B and C present the ³H-thymidine incorporation of representative normal peripheral blood mononuclear cells after exposure to a monofilament nylon suture (A), chromic gut suture (B) or plain gut suture (C). ³H-thymidine incorporation increased the most following exposure to nylon suture coated with αCD3 and αCD28 monoclonal antibodies.

FIG. 2A and B show the proliferative response of peripheral blood mononuclear cells to monofilament nylon suture either uncoated (A) or coated with αCD3/αCD28 monoclonal antibodies (B).

FIG. 3 presents the proliferative responses on day 6 of LNMCs and PBMCs from 11 patients with head and neck squamous cell carcinoma and PBMCs from 6 normal subjects. PBMC indicates peripheral blood mononuclear cells and LNMC indicates lymph node mononuclear cells. The large bar is the PBMC mean, the large circle is the normal control PBMC mean and the large triangle is the LNMC mean.

FIG. 4 shows the phenotype of T-cells on days 0 and 6 after anti-CD3/anti-CD28 nylon suture stimulation. All data are given as the mean ± the standard deviation. The dagger indicates a significant increase on day 6.

FIG. 5 shows cytokines present on day 6 following stimulation of patients with HNSCC with αCD3/αCD28 nylon suture or plastic. The dagger represents a significant increase above untreated controls. The double dagger represents a significant increase above coated plastic stimulation.

FIG. 6 shows cytokines present on day 6 after stimulation of controls with αCD3/αCD28 nylon suture or plastic. The dagger represents a significant increase above untreated controls. NT indicates an untested value.

FIG. 7 demonstrates the cytotoxicity of αCD3/αCD28 stimulated lymph node lymphocytes and peripheral blood lymphocytes to autologous tumor cells. The effector:target (E:T) ratio is presented on the X-axis and represents the ratio of stimulated effector cells to tumor cells.

FIG. 8 shows the proliferation of lymph node lymphocytes along nylon (A) and polyester (B) suture coated with anti-CD3 and anti-CD28 monoclonal antibody.

FIG. 9 demonstrates the proliferation of peripheral blood lymphocytes in response to nylon suture coated with anti-CD3 and anti-CD28 monoclonal antibodies and polyester suture coated with interferon-gamma.

FIG. 10 shows that anti-interferon-PE binds to polyester suture coated with interferon-gamma (B), but not to control polyester suture (A).

### DETAILED DESCRIPTION OF THE PREFERRED EMBODIMENT

The present invention is based on the discovery that suture can be coated with one or more biologically active compounds, for example antibodies (Abs). The resulting coated suture is a “bioactive surgical suture.” The compounds maintain their biological activity when present on the suture.

The term “antibody” is used in the broadest sense possible and covers, without limitation, monoclonal antibodies (including agonist, antagonist, and neutralizing antibodies), antibody compositions with polyepitopic specificity, single chain antibodies, and fragments of antibodies. The term “monoclonal antibody” as used herein refers to an antibody wherein the individual antibodies comprising the population are identical except for possible naturally occurring mutations that may be present in minor amounts.

“Coated suture” refers to a piece of surgical suture that has been coated with one or more biologically active compounds according to the methods of the present invention. The suture is “coated” if one or more molecules of a biologically active compound are bound to the suture. Preferably, the biologically active compound is non-covalently bound to the suture.

The suture used in the present invention may be made of any material known in the art. The suture material is preferably vicryl, polyester, nylon, polyglactin, chromic gut, plain gut or silk. More preferably the suture material is nylon. The suture may be of any length. In one embodiment the suture is 1 cm in length. Preferably the suture is long enough to reach from the site of placement to the surface of the skin of the organism in which it is placed.

The suture may be of any thickness. Preferably the suture is 3-0 suture.

One aspect of the present invention provides a method of coating suture with one or more biologically active compounds. The “biologically active” or “bioactive” compounds used in the present invention may be any compounds that produce a biological response when administered to an organism. There is no limitation on the type of molecules or compounds that may be used. For example, the biologically active compounds may be small molecule organic or inorganic molecules or compounds, nucleic acids, peptides, antibodies or any combination thereof. Preferably the biologically active compounds are peptides. More preferably the biologically active compounds are antibodies, such as anti-CD3, anti-CD28, 41-BB, anti-CD40L and anti-CD40. Even more preferably the biologically active compounds are anti-CD3 and anti-CD28 monoclonal antibodies. Several non-limiting examples of other types of biologically active molecules or compounds that may be used are antibiotic, anti-fungal, anti-malarial, anti-tubercular, anti-viral and anti-microbial compounds, cephalosporins, aminoglycosides, penicillin, defensins, chemokines, cytokines, interleukin-2, interleukin-12, interferon-gamma, epitope specific sequences such as p53, E75, E6 and E7, and EBV associated antigens.

In the preparation of coated suture, the suture is contacted with the biologically active compounds. In the preferred embodiment, lengths of suture are incubated in a solution that contains the biologically active compounds. The biologically active compounds are preferably present in the solution at a concentration at which they remain soluble. More preferably the biologically active compounds are present in the solution at a concentration of from 0.01 to 500 μg/ml. Even more preferably the concentration of the biologically active compounds is from 1 to 50 μg/ml and yet more preferably from 2 to 10 μg/ml.

In the preferred embodiment the biological activity of the biologically active compounds is maintained in the solution. The solution is preferably a buffer. In one embodiment the suture is incubated in phosphate buffered saline (PBS) containing the desired biologically active compound. More preferably the solution is Ca²⁺ and Mg²⁺ free PBS. One skilled in the art will recognize that the amount of buffer used will vary depending on the size of the container in which the incubation takes place, the total length of suture being incubated and the desired concentration of the biologically active compounds in the buffer. Preferably from 0.1 to 10 ml of buffer is used for every 1 cm of suture incubated, more preferably from 1 to 5 ml of buffer for every 1 cm of suture and even more preferably 2 ml of buffer for every 1 cm of suture.

The suture is incubated in the buffer containing the biologically active compounds for a length of time sufficient to allow the compound to adhere to the suture. Preferably the incubation is continued for from 0.1 to 120 hours. More preferably the incubation is continued for from 1 to 36 hours and even more preferably for from 4.5 to 18 hours.

In the preferred embodiment the incubation is carried out at a temperature at which the biologically active compound is the most thermally stable. This temperature may readily be determined by one skilled in the art. The incubation preferably takes place at from 10 to 90° C., more preferably from 30 to 50° C. and even more preferably at 37° C.

The incubation may be carried out in any container capable of holding the suture and the buffer containing the biologically active compounds. Preferably the container is made of a material that does not interact with the biologically active compounds. More preferably the incubation is carried out in a tissue culture dish. One skilled in the art will recognize that the preferred tissue culture dish will be chosen based on the amount of suture that is to be coated with the biologically active compound. For example, single, 1 cm long pieces of suture are preferably incubated in single wells of 96 well plates, while from 3 to 6, 1 cm long pieces of suture may be incubated in a single well of a 24 well tissue culture plate. Longer pieces of suture are incubated in appropriately larger containers.

In the preferred embodiment, the coated suture is washed in buffer that does not contain the biologically active compounds before use. Preferably the coated suture is washed from 1 to 10 times in an excess of PBS, more preferably from 2 to 5 times and even more preferably 3 times.

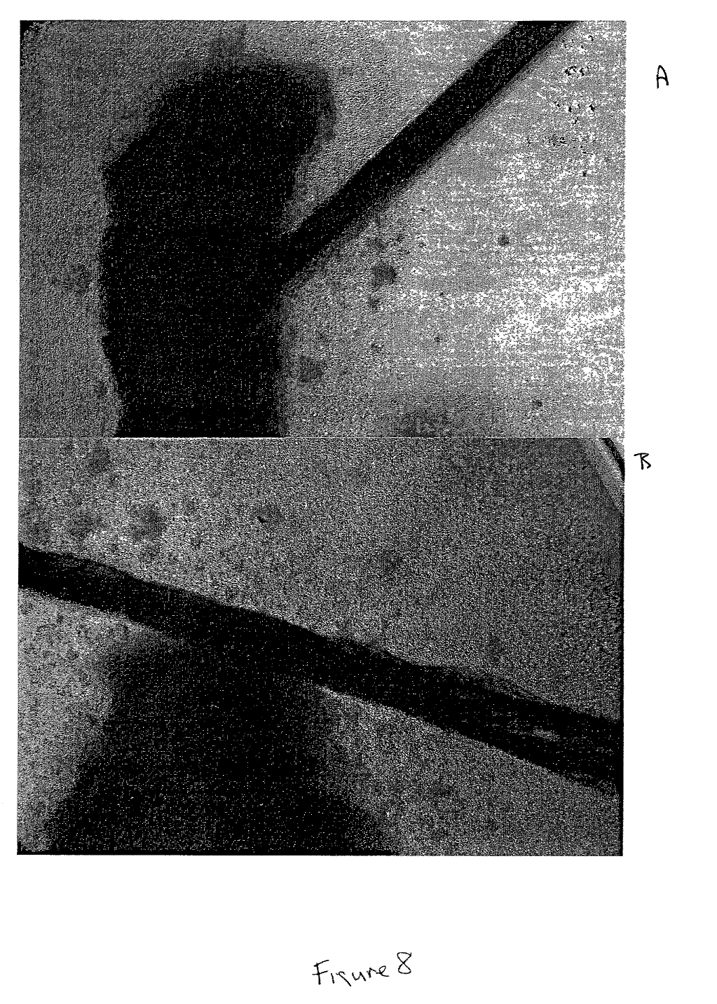

In one aspect of the invention the coated suture is used in vitro or ex vivo. The coated suture is brought into contact with cells that are responsive to the biologically active compounds on the suture. In one embodiment, suture coated with one or more immuno-stimulating molecules or compounds is used ex vivo under sterile conditions to expand immunocompetent cells. For example, a lymph node may be removed from a patient and contacted with a suture that is coated with molecules that stimulate lymphocytes. FIG. 8 shows the proliferation of lymph-node lymphocytes following placement of a suture coated with anti-CD3 and anti-CD28 monoclonal antibodies in a lymph node ex vivo. The lymphocytes may then be expanded and the expanded cells may be used for adoptive cellular therapy to treat cancer or other illnesses, such as infection, immunodeficiencies or chronic illnesses.

In another aspect of the invention, the coated suture is placed into an organism to provide in vivo therapy. Preferably the organism is a mammal and even more preferably a human.

In one embodiment the organism is a human patient. The coated suture may be placed in the patient as part of a course of treatment for a disease or disorder. Alternatively, the coated suture may be placed in the patient to prevent the development of a disease or disorder. The coated suture may also be placed in a human patient who is receiving an organ transplant.

In one embodiment the human patient has cancer. In particular, the human patient may have head and neck cancer, more particularly squamous cell carcinoma.

The coated suture may be placed in an organism by any method known in the art. In the preferred embodiment a hollow needle with a trochar is passed into the desired location in the organism. One skilled in the art will recognize that the length of the needle will vary depending on the depth at which the coated suture is to be placed. The trochar is then removed and the suture is passed down the barrel of the needle to the end. The needle is then removed over the coated suture, leaving the coated suture in the desired position. The coated suture preferably extends from the desired position to the surface of the skin.

One or more coated sutures may be placed in the same organism by the method of the present invention. In particular the desired biological activity in the organism may be modified by adding or removing coated sutures.

The coated suture may remain in the organism for any length of time. However, it is anticipated that the biological activity will decrease over time once a coated suture has been placed in an organism. Thus to maintain the desired biological activity the old coated suture may be removed and replaced with new coated suture at regular intervals. This may be accomplished without subjecting the patient to a major surgery. The coated suture may also be removed if the organism experiences an adverse reaction to the coated suture or if the coated suture has an unforeseen biological activity.

In the preferred embodiment, the location of the coated suture in the organism is confirmed during or after placement. For example, manual palpation, ultrasound, or CT scan may be used to confirm the location of the coated suture. In one embodiment the distal end of the coated suture is modified to be radio-opaque, thus allowing the location of the suture to be determined by radiograph.

The proximal end of the coated suture may be secured to the skin, for example with a piece of tape or a bandage. In this embodiment the coated suture may be easily removed by pulling on the proximal end of the suture. In another embodiment the coated suture is removed by passing the proximal end of the coated suture into a sterile 18-20 gauge spinal needle which is then passed over the coated suture to core it out.

The suture of the present invention is coated with a particular compound and placed in a specific location such that it produces a desired biological response. For example, the coated suture may find use in modulating the immune system of a patient in which it is placed. Thus the suture may be coated with an immuno-modulating agent and placed in a location in the body where the immuno-modulating agent would be expected to have a biological activity, for example in the lymph node. In another examples the suture is coated with anti-microbial compounds, such as antibiotics and placed in a wound to prevent infection. In a further example the suture may be coated with a compound that facilitates wound healing and placed into a wound.

In particular the coated suture of the present invention may find use in stimulating the immune system of a human patient suffering from a disease that is characterized by suppression of the immune system. More particularly the suture may find use in treating cancer patients. For example, immuno-modulating sutures may be placed in the regional lymph nodes of cancer patients to stimulate cytotoxic T lymphocytes, helper T lymphocytes, Natural Killer cells, dendritic cells and other immune competent cells in the fight against cancer. Immuno-modulating coated sutures have been shown to generate a T_{H}1 type immune response that is effective in the rejection of multiple cancer varieties (see Example 3).

In one embodiment, immuno-modulating coated suture is used to treat a cancer patient by placing the suture by injection into identified lymphatics where they stimulate an anti-cancer response. Preferably the coated suture comprises one or more pieces small enough to be injected. One advantage of this method is that it may be performed in vivo and thus does not require the labor intensive step of adoptive cellular therapy and cellular expansion. Additionally, the risks of contamination and infection are significantly reduced compared to in vitro cellular expansion and infusion.

In another embodiment immuno-modulating coated sutures of the present invention are placed in the regional lymph nodes of cancer patients prior to surgery. This stimulates the immune system and enhances a population of immunocompetent cells that are immune specific for the particular cancer being treated. The immunocompetent cells are harvested from the lymph node following surgical removal and are expanded ex vivo using any method known in the art, for example αCD3 and IL-2 treatment or incubation with αCD3 and αCD28 coated beads. The expanded cells are then reinfused into the patient to help fight the cancer.

The immuno-modulating coated suture of the present invention may also find use when combined with tumor specific peptide in a vaccine regimen. Patients are immunized according to a standard immunization protocol. In addition the immune system is stimulated by placing one or more of the coated sutures of the present invention, providing for a stronger anti-cancer immune response.

In another embodiment, sutures coated with specific peptides are placed into a surgical wound following resection. The coated sutures stimulate the residual memory cells present in the wound area, which leads to the death of any residual microscopic cancer.

In another embodiment the immuno-modulating coated suture is used to suppress the immune system. A suture is coated with αCD3 by the method of the present invention. αCD3 is commonly used in organ transplantation to prevent host rejection of the donor organ. At the time of transplant surgery the anti-CD3 coated suture is strategically placed into the donor organ bed or into the regional lymphatics to cause a local or regional immunosuppressed environment and thus reduce the chance of donor organ rejection.

### EXAMPLES

### Example 1

Monofilament nylon suture is coated with αCD3 and αCD28 antibodies. αCD3 antibody is added to Ca²⁺ and Mg²⁺ free PBS to a concentration of 2 μg/ml. An equivalent amount of αCD28 antibody is then added to the solution. A 5 cm suture is incubated in 1 ml of the antibody-containing PBS solution in one well of a 24 well tissue culture plate. The incubation is continued for 6 hours at 37° C.

### Example 2

Chromic gut or plain gut suture is coated with αCD3 and αCD28 antibodies. αCD3 antibody is added to Ca²⁺ and Mg²⁺ free PBS to a concentration of 10 μg/ml. An equivalent amount of αCD28 antibody is then added to the solution. A 5 cm suture is incubated in 1 ml of the antibody-containing PBS solution in one well of a 24 well tissue culture plate. The incubation is continued for 6 hours at 37° C.

### Example 3

T-cells can be stimulated ex vivo with αCD3/αCD28 monoclonal antibodies. The resulting cells have been used in adoptive immunotherapy trials. The coated surgical suture of the present invention was used as a novel carrier for αCD3/αCD28 monoclonal antibodies (MoAbs). The coated suture allows in vivo immune stimulation and bypasses the need for in vitro expansion and re-infusion. To test the properties of αCD3/αCD28 coated suture the activation of peripheral blood mononuclear cells (PBMCs) from normal patients was measured. In addition, PBMCs and lymph node mononuclear cells (LNMCs) from patients with advanced head and neck squamous cell carcinoma (HNSCC) were incubated in vitro with the antibody-coated suture and the patients' responses were measured. Patients with HNSCC were chosen because of the known immunosuppression that exists in this patient population. The immune responses measured were proliferation, cytokine production and cellular phenotype.

Peripheral blood was drawn from healthy volunteers or patients with HNSCC before surgery. Blood was suspended in an equal volume of PBS and PBMCs were isolated by centrifugation over a Ficoll-Hypaque (Pel-Freez, Brown Deer, Wis.) density gradient for 10 minutes at 2400 rpm. PBMCs were cultured at a density of 1×10⁵ in 200 μl of culture media in 96-well flat-bottom plates (Costar, Cambridge, Mass.). Culture media consisted of RPMI 1640 (Gibco, Paisley, Scotland) supplemented with 10% fetal calf serum (Hyclone), 2 mmol/L glutamide (Gibco), 100 U/ml penicillin (Gibco), 100 μg/ml streptomycin (Gibco) and 100 U/ml amphotericin B (Gibco).

Lymph nodes from HNSCC patients were harvested at the time of surgery. The mean age of all the patients studied was 51.7 years, with a range of 38 to 65 years. All patients had advanced stage III or IV HNSCC. The site of the HNSCC primary cancers included the oropharynx, the larynx, the oral cavity and an unknown primary cancer. Harvested lymph nodes were immediately placed in balanced salt solution with 20% heat inactivated fetal calf serum (Hyclone, Logan, Utah), a combination of 1% penicillin potassium and streptomycin sulfate and 1% amphotericin B. Lymph nodes were then minced, filtered through a nylon mesh and washed twice in a balanced salt solution with 5% fetal calf serum, 1% penicillin-streptomycin and 1% amphotericin B. Lymph node mononuclear cells (LNMCs) were obtained by Ficoll-Hypaque density gradient centrifugation. Only pathologically confirmed negative lymph nodes were used in this study. For phenotyping and cytokine quantification, 7.5×10⁵ PBMCs or LNMCs were cultured in 2.0 ml of culture media in 24 well tissue culture plates for 6 days. All cultures were maintained at 37° C. in a 5% carbon dioxide atmosphere.

The immune response of cells was measured over an 8-day period. On days 2, 4, 6 and 8, cell cultures were pulsed with 7.4×10⁴ Bq of ³[H]-thymidine (tritium-thymidine) for 4 hours and harvested onto glass fiber disks using a cell harvester (Cambridge Technology, Cambridge, Mass.). The glass fiber disks were placed in vials containing 6 ml of scintillation cocktail and counted in a scintillation counter (Beckman, Fullerton, Calif.).

For phenotypic analysis, cell suspensions were prepared from LNMC or PBMC cultures on days 0 and 6 after incubation. The cells were stained with anti-CD3-phycoerythrin (PE), anti-CD4-PE, anti-CD8-PE, anti-CD28-PE or anti-CD45RO-PE (PharMingen, San Diego, Calif.). The expression of surface markers was measured by flow cytometry (FACScan, Becton Dickinson, San Jose, Calif.).

For cytokine analysis, cell culture supernatants were harvested on days 2, 4 and 6. The quantities of IL-2, IL-4, TNFα, IL-12 and IFN-γ present in the supernatant was determined by enzyme-linked immunosorbent assay (R&D Systems, Minneapolis, Minn.).

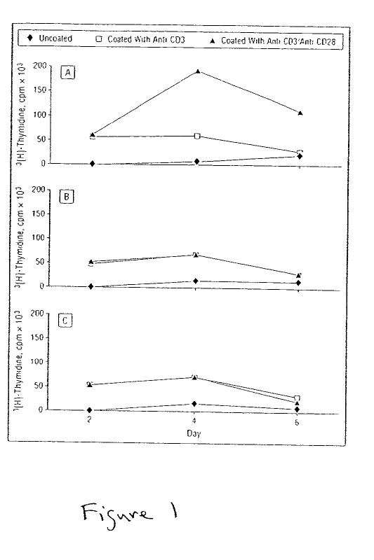

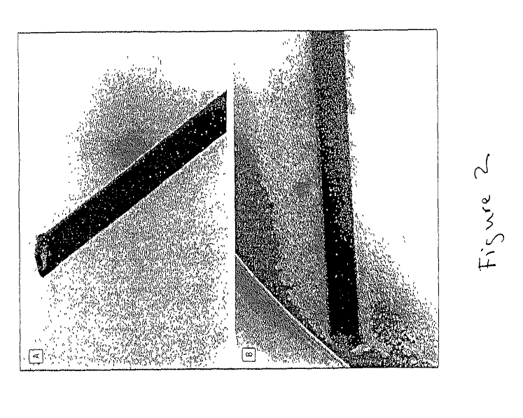

Suture coated with αCD3 was prepared by incubating sterile suture in PBS containing αCD3 (Caltag Corp., Burlingame, Calif.) at various concentrations for 18 hours at 37° C. The suture was washed 3 times in phosphate buffered saline (PBS) before culturing with mononuclear cells. Monofilament nylon, chromic gut and plain gut sutures (Ethicon Inc., Somerville, N.J.) were found to consistently activate T cells after coating with αCD3 MoAbs (FIG. 1 A-C). Uncoated suture did not stimulate normal PBMCs (FIG. 2A). All 3 suture types stimulated maximal proliferation on day 4 (FIG. 1 A-C).

Because αCD3-coated nylon, chromic gut and plain gut sutures stimulated consistently, the efficacy of coating with αCD3/αCD28 was examined (FIG. 1 A-C). Suture coated with αCD3/αCD28 was prepared by incubating sterile suture in PBS at various concentrations and ratios of the αCD3 and αCD28 (private gift) antibodies for 18 hours at 37° C. The optimal coating condition was achieved by incubating nylon suture with 2 μg/ml αCD3/αCD28 (1:1). The types of suture studied included nylon, polyglactin, chromic gut, plain guy and silk (Ethicon Inc, Somerville, N.J.). The suture was washed 3 times in phosphate buffered saline (PBS) before culturing with mononuclear cells. Nylon suture exhibited the strongest MoAb carrier function (FIG. 2B). Proliferation of PBMCs induced by αCD3/αCD28-coated nylon suture exceeded that by αCD3-coated suture (FIG. 1A).

In order to compare the stimulating capacity of coated suture to a known αCD3/αCD28 carrier, proliferative responses of PBMCs and LNMCs from patients with HNSCC were measured after exposure to αCD3/αCD28 coated suture or αCD3/αCD28 coated tissue culture plastic. Tissue culture plastic is a recognized carrier of αCD3/αCD28 monoclonal antibodies. Tissue culture plastic was coated with 100 μl of varying concentration s of αCD3 or αCD3/αCD28 and incubated at 37° C. for 18 hours. Plates were washed three times with PBS before use in the assays.

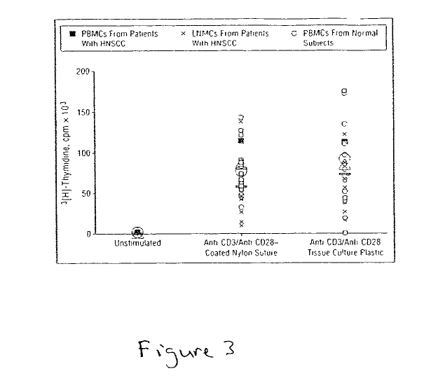

Proliferative responses of LNMCs and PBMCs peaked on day 6 for both coated suture and tissue culture plastic and no differences were observable between the two carriers (FIG. 3). Normal donor PBMCs were also stimulated using αCD3/αCD28 on both carriers and similar results were obtained, with no significant difference between the tissue culture plastic and the coated suture (FIG. 3).

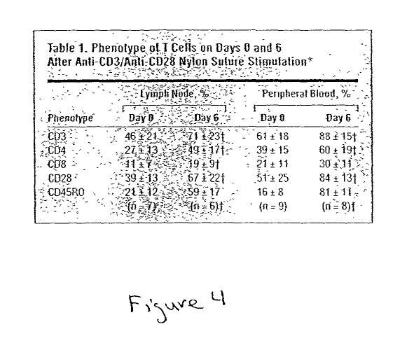

In order to identify the T-cell population from the lymph node that is expanded after αCD3/αCD28 suture stimulation, T-cells were characterized before activation and on day 6 after stimulation. Lymph node mononuclear cells stimulated with αCD3/αCD28 coated suture revealed significant enhancement in all T-cell subpopulations. In particular, a significant increase occurred in the CD3, CD4, CD8, CD28 and CD45RO populations as measured by flow cytometry (FIG. 4). A similar increase in T-cells was observed after PBMC stimulation, with significant increases observed by day 6 in the CD3, CD4, CD28 and CD45RO populations. The largest increase was noted in the CD45RO or memory cell population of T-cells. Stimulation with αCD3/αCD28 coated plastic did not produce adequate expansion of peripheral blood and lymph node T-cells to allow phenotyping.

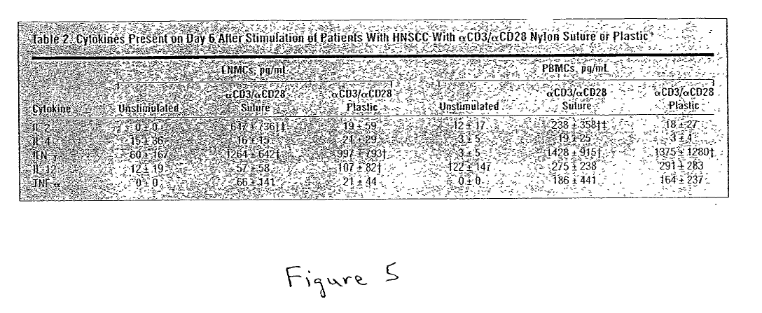

The immunologic response enhanced by αCD3/αCD28 coated suture and plastic was further characterized by harvesting cell culture supernatants following stimulation. Cell culture supernatants were harvested on day 6 following stimulation and the quantity of IL-2, IL-4, IL-12, IFN-γ and TNFα was measured by enzyme-linked immunosorbent assay. Unstimulated cultures revealed minimal expression of cytokines (FIG. 5). Stimulation of LNMCs with αCD3/αCD28 coated suture enhanced the secretion of IL-2 significantly compared to both unstimulated cells and cells stimulated with αCD3/αCD28 coated plastic (FIG. 5). Stimulation of LNMCs with αCD3/αCD28 coated suture induced significantly higher levels of IFN-γ production than those seen in unstimulated controls, as did αCD3/αCD28 coated plastic (FIG. 5).

These results indicate that stimulation with αCD3/αCD28 suture resulted in a T_{H}1 cytokine expression pattern in the LNMCs studied. Stimulation of PBMCs with coated suture also induced significantly higher production of IL-2 and IFN-γ compared to unstimulated controls (FIG. 5). As can be seen in FIG. 5, suture stimulated PBMCs also produced significantly higher levels of IL-2 than coated plastic stimulation. This indicates that stimulation with αCD3/αCD28 suture produced a T_{H}1 cytokine expression pattern in PBMCs as well.

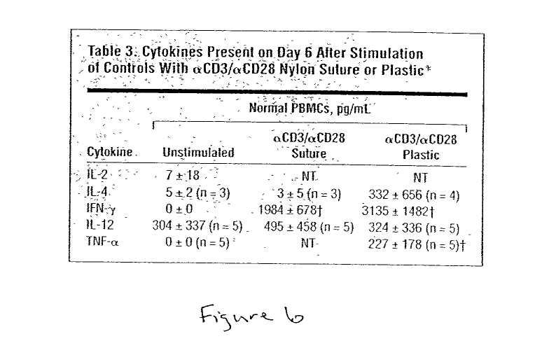

Stimulated cytokine expression was also measured in normal donor PBMCs in vitro after 6 days (FIG. 6). αCD3/αCD28 stimulation significantly increased expression of IFN-γ above unstimulated control levels. Coated plastic stimulation increased expression of both IFN-γ and TNFα levels above those seen in unstimulated controls (FIG. 6). There was no significant difference in the quantity of IFN-γ secreted after coated suture stimulation or coated plastic stimulation.

The induction of T_{H}1 cytokines (IL-2 and IFN-γ) by αCD3/αCD28 coated suture is significant. T_{H}1 cytokines have been associated with cytotoxic immune responses against cancer. There are at least 2 active FDA approved adoptive immunotherapeutic trials using αCD3/αCD28 coated beads as immunostimulants for the treatment of human immunodeficiency virus and advanced end-stage solid cancers. αCD3/αCD28 coated surgical suture as a carrier will allow cellular activation to be performed in vivo, bypassing the need for ex vivo expansion and reinfusion. Targeted areas could be the primary cancer site or regional lymph nodes.

### Example 4

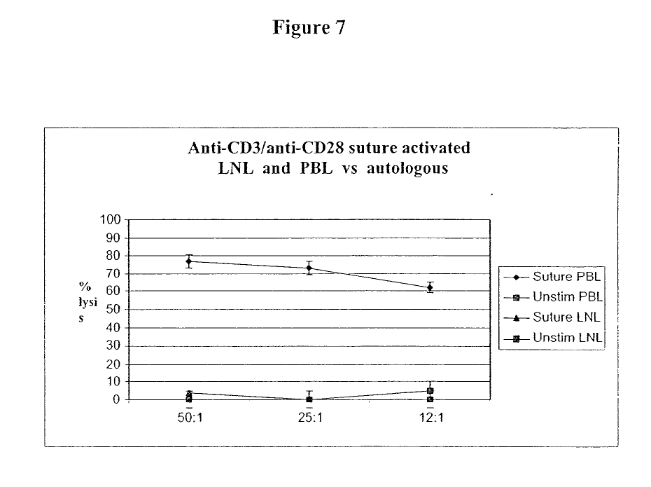

The antibody-coated sutures of the present invention may be used effectively to kill tumor cells from a patient with head and neck squamous cell carcinoma (HNSCC). A cytotoxicity assay was performed using lymph node lymphocytes (LNL) or peripheral blood lymphocytes (PBL) against autologous tumor. LNL and PBL from a HNSCC patient were stimulated with suture coated with αCD3/αCD28 according to the method of the present invention. Stimulated cells were used in a ⁵¹Cr cytotoxicity assay to measure their ability to kill autologous tumor cells. Briefly, tumor cells from the patient's own tumor were labeled with ⁵¹Cr. The tumor cells were then contacted with the stimulated LNL or PBL cells and the resulting ⁵¹Cr release was measured. Stimulated PBL were able to kill 77%, 73% and 62% of all tumor cells at 50:1, 25:1 and 12:1 ratios of effector or activated T-cells to tumor cells (effector:target; E:T ratio) respectively (FIG. 7). Stimulated LNL were able to lyse 4% of tumor cells at a 50:1 E:T ratio (FIG. 7).

### Example 5

Antibody coated suture is used in the treatment of residual cancer present in the neck as a large palpable mass. The tumor is localized with 1% lidocaine. An 18-20 gauge spinal needle approximately 6-8 inches in length is placed into the center of the tumor. Placement into a large tumor is confirmed by simple palpation. In smaller tumors placement is confirmed by ultrasound, CT scan or plan radiograph. The trochar is removed and αCD3/αCD28 coated suture is passed down the barrel of the needle. The needle is then removed over the suture. The suture has thus been placed without any abrading through the skin or soft tissue. The suture is secured to the skin with a piece of tape or bandage.

### Example 6

Immunomodulating suture of the present invention is used in the treatment of a patient with head and neck cancer. First, a suture coated with human defensin is placed into the palpable regional lymph node. This attracts immature CD34+ antigen presenting cells that are believed to be immunosuppressive. After several days of attracting these cells to the area within the lymph node near the suture, the defensin coated suture is removed and replaced with an αCD3/αCD28 coated suture. This suture stimulates the immature cells to become more mature antigen presenting cells. The αCD3αCD28 suture is removed after several days and replaced with a suture coated with antigens specific to the type of cancer found in the patient. This suture produces an immune specific response. A second suture is then added to help boost the specific immune response. The second suture is coated with αCD3/αCD28 antibody. This combination of immuno-modulating sutures stimulates the lymph node lymphocytes to kill the cancer.

### Example 7

The therapy presented in Example 5 may be followed by adoptive immunotherapy. The patient suffering from head and neck cancer is taken to surgery and a neck dissection and tumor resection is performed. During the neck dissection the lymph nodes that were the site of the suture placement in Example 5 are harvested and the immuno-stimulated lymphocytes are expanded in vitro. The immuno-modulating suture will have increased the population of anti-cancer lymphocytes in the lymph node, thus providing more cells for the in vitro expansion. The expansion is carried out using standard protocols well known in the art. The expanded population of lymphocytes will have been exposed to the tumor and will have an enhanced ability to kill the cancer. The expanded anti-cancer cells are then infused into the patient.

### Example 8

Patients with a cancer that presents specific tumor antigens are vaccinated with antigen coated suture. The vaccination may be done in conjunction with the use of immuno-stimulating suture as described above. Epstein Bar Virus (EBV) associated proteins are commonly found on the tumor cells of patients with nasopharyngeal carcinoma. P53, E75, E6 and E7 antigens have also been associated with head and neck carcinoma and other cancers. Suture coated with one or more peptides selected from the group consisting of EBV peptide, p53, E75, E6, E7 and other cancer specific peptides is placed in the regional neck lymph node before, during and after standard treatment with radiation therapy. The peptide-coated suture stimulates the immune system to kill any cancer that develops after treatment and thus “vaccinates” the patient. Simultaneously the patient is treated with immuno-modulating suture as described in Example 5. The patient may receive intermittent booster immuno-modulating suture during the course of the year.

### Example 9

Following standard surgical resection of a tumor in a patient with head and neck cancer, immuno-modulating sutures are place directly in the wound. At least one suture is coated with tumor specific peptides and at least one suture is coated with αCD3/αCD28. The sutures are placed directly in the floor of the wound and the ends of the sutures are brought out the incision site. The sutures located in the wound bed stimulates the residual memory cells present in the wound area and enhance the anti-tumor response of the immune system as healing occurs.

### Example 10

Following standard surgical resection of a tumor in a patient with head & neck cancer, sutures coated with biologically active compounds are place directly in the wound. Immuno-stimulating suture has been shown to induce a Th1 type of immunological response. This response is effective at killing foreign bodies, such as bacteria, that may contaminate a surgical wound. Suture coated with αCD3/αCD28 is placed in the desired location in the surgical bed and brought out the incision site in the same manner as a surgical drain. Additional sutures coated with anti-microbial peptides, such as defensins, or antibiotics may be placed in the surgical bed as well.

### Example 11

αCD3 is presently used to reduce transplant rejection and is administered by systematic infusion. An immuno-modulating suture of the present invention may be used to specifically suppress the lymphatic most commonly associated with a particular organ rejection. At the time of placing a donor kidney into the recipient's pelvis, αCD3 coated suture is placed into the pelvic lymph nodes or the organ bed. This causes regional immunosuppression and reduces the incidence of host rejection of the donor organ.

### Example 12

Severe combined immune-deficiency (SCID) mouse model for testing bioactive suture.

A first group of SCID mice is inoculated subcutaneously with head and neck squamous cell carcinoma (HNSCC) take from cancer patient A in the flank, thigh or back. This group of mice functions as a cancer control group.

Lymph node tissue harvested from cancer patient A is implanted into the flank, thigh or back of a second group of SCID mice. This group of mice functions as a non-cancer control group.

A third group of SCID mice has both HNSCC and lymph node tissue from patient A implanted into the flank, thigh or back. The cancerous tissue and lymph node tissue are placed adjacent to one anther. This third set of mice represents the baseline immune response of lymph node lymphocytes to HNSCC from cancer patient A. The cancer and lymph node are allowed to grow for 3-5 days in vivo proximal to each other. Then on day 6 αCD3/αCD28 coated suture is placed into the lymph node bed using the suture placing technique described above. αCD3/αCD28 suture is left in place for the next 10 days and the anti-cancer response is measured by sequential measurement of tumor size. Animals are sacrificed at selected time points and the anti-cancer response is measured histologically. Immune responsive cells are phenotyped by flow cytometry and the immune environment is characterized by measuring the cytokine profile using intra-cellular stains and enzyme linked immunosorbent assay techniques well known in the art.

In addition to using suture coated with αCD3/αCD28, suture coated with interleukin-2, interferon-gamma, interleukin-12, 4-1-BB, anti-CD40, anti-CD40L, or p53, E75, E6, E7 or EBV antigens, is used as an immune stimulant individually or in combinations.

A fourth group of SCID mice with both HNSCC and lymph node tissue from patient A implanted adjacent to one another in the flank, thigh or back is treated with uncoated suture. This group of mice functions as a non-stimulating suture control group. The immune response in this group is analyzed in the same way as in the group receiving the stimulating suture.

### Example 13

Lymph nodes were removed from a patient with advanced stage head and neck squamous cell carcinoma. Sutures coated with biologically active compounds were placed in the lymph node and the proliferation of lymph node lymphocytes was observed. The proliferation of lymph node lymphocytes along anti-CD3/anti-CD28 monoclonal antibody coated (a) nylon and (b) polyester suture is shown in FIG. 8.

### Example 14

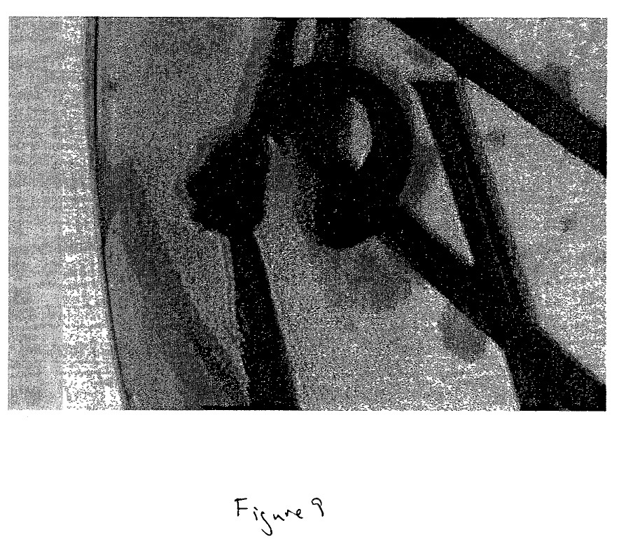

The response of peripheral blood lymphocytes to suture coated with biologically active compounds was measured in vitro. FIG. 9 shows that the peripheral blood lymphocytes proliferated in response to nylon suture coated with anti-CD3 and anti-CD28 monoclonal antibody and polyester suture that was coated with interferon-gamma.

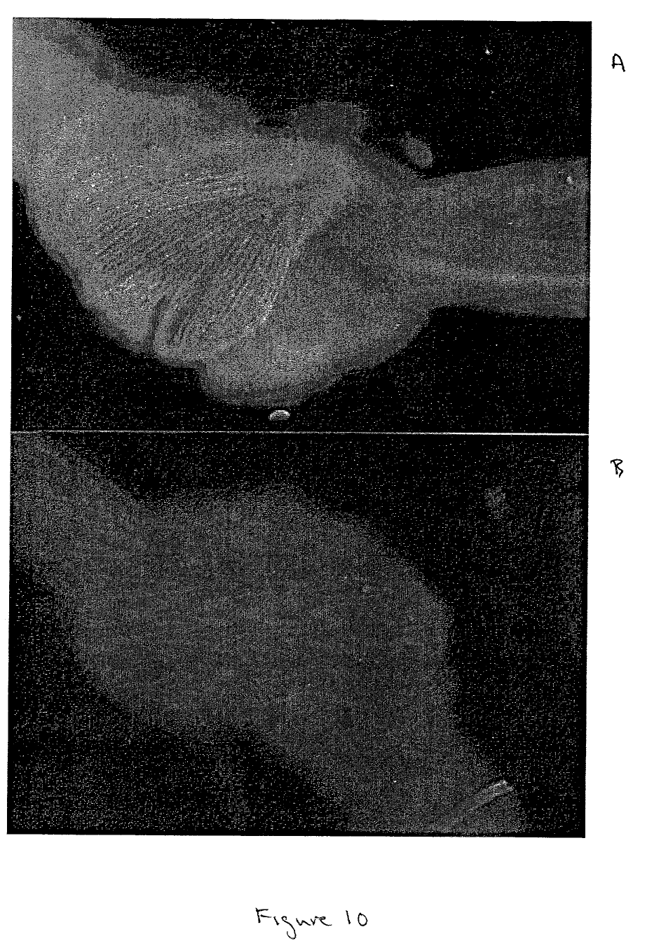

The binding of interferon-gamma to 5-0 polyester suture was confirmed by immunofluorescence. FIG. 10 shows that anti-interferon-PE bound to interferon-gamma coated polyester suture (B) but not to control polyester suture (A).

### Conclusion

As can be seen from the foregoing discussion, the coated sutures of the present invention can be used for long-term delivery of a variety of different biologically active compounds in a wide variety of local and systemic environments. As such, those of ordinary skill in the art will appreciate upon access to the present disclosure that a large number of specific treatments can be carried out using these sutures, in addition to those specifically exemplified herein.

What is claimed is:

1. A method of coating surgical suture with a biologically active compound comprising:
placing a length of surgical suture into a solution comprising the biologically active compound; and
incubating the suture in the solution until the biologically active compound is bound to the suture.

2. The method of claim 1 wherein the surgical suture is selected from the group consisting of polyester suture, vicryl suture, nylon suture, plain gut suture, polyglactin suture, chromic gut suture and silk suture.

3. The method of claim 1 wherein the solution is phosphate buffered saline.

4. The method of claim 3 wherein the phosphate buffered saline is Ca²⁺ and Mg²⁺ free.

5. The method of claim 1 wherein the incubation is carried out for about 18 hours.

6. The method of claim 1 wherein the incubation is carried out at about 37° C.

7. The method of claim 1 wherein the solution comprises about 2 μg/ml of the biologically active compound.

8. The method of claim 1 wherein the biologically active compound is defensin.

9. The method of claim 1 wherein the biologically active compound is an antibody.

10. The method of claim 9 wherein the antibody is a monoclonal antibody.

11. The method of claim 9 wherein the biologically active compound is selected from the group consisting of αCD3 and αCD28 antibodies.

12. The method of claim 1 wherein the suture is coated with two or more biologically active compounds.

13. The method of claim 12 wherein the suture is coated with αCD3 and αCD28 antibodies.

14. The method of claim 1 further comprising modifying the suture such that it is visible on a radiograph.

15. A method of placing a surgical suture coated with one or more biologically active compounds into a patient comprising:
passing a needle with a trochar into the desired location in the patient;
removing the trochar;
passing the suture down the barrel of the needle;
removing the needle over the suture while leaving the suture in position.

16. The method of claim 15 wherein the position of the needle is determined prior to removing the needle.

17. The method of claim 16 wherein the position of the needle is determined by a technique selected from the group consisting of manual palpation, ultrasound, CT scan and radiography.

18. The method of claim 15 additionally comprising securing the proximal end of the suture to the skin of the patient.

19. The method of claim 15 wherein the suture is labeled such that it is visible on a radiograph.

20. The method of claim 19 further comprising determining the location of the suture with a radiograph.

21. A method of reducing rejection of transplanted organs comprising placing a suture coated with an immunosuppressive compound into the organ bed or regional lymphatics of a patient receiving a donor organ.

22. The method of claim 21 wherein the suture is coated with an immunosuppressive antibody.

23. The method of claim 22 wherein the suture is coated with μCD3 monoclonal antibody.

24. The method of claim 21 wherein the suture is placed in the pelvic lymph node of the patient.

25. A surgical suture coated with at least one biologically active compound.

26. The suture of claim 25 wherein the biologically active compound is selected from the group consisting of antibiotic, anti-fungal, anti-malarial, anti-tubercular, anti-viral and anti-microbial compounds.

27. The suture of claim 25 wherein the biologically active compound is defensin.

28. The suture of claim 25 wherein the biologically active compound is selected from the group consisting of the epitope specific sequences p53, E75, E6, E7 and EBV associated antigens.

29. The suture of claim 25 wherein the biologically active compound is an antibody.

30. The suture of claim 29 wherein the antibody is selected from the group consisting of anti-CD3, anti-CD28, anti-CD40, anti-CD40L and 41-BB.

31. The suture of claim 25 wherein the suture is non-covalently coated with the biologically active compound.

32. The suture of claim 31 wherein the suture is coated with anti-CD28 and anti-CD3 antibodies.

33. The suture of claim 25 wherein the suture is modified such that it is visible on a radiograph.

34. A method of treating a patient suffering from a disease that is characterized by suppression of the immune system comprising placing a suture coated with one or more immuno-stimulating compounds into a lymph node in the patient.

35. The method of claim 34 wherein the suture is coated with anti-CD3 and anti-CD28.

36. The method of claim 34 wherein the disease is cancer.

37. A method of treating a patient suffering from head and neck cancer comprising:
placing a first suture coated with human defensin into a palpable regional lymph node; and
placing a second suture coated with anti-CD3 and anti-CD28 antibodies into a palpable regional lymph node.

38. The method of claim 37 wherein the first suture is removed prior to placing the second suture.

39. The method of claim 37 additionally comprising placing a third suture in the palpable regional lymph node, said third suture being coated with antigens specific to the type of cancer that the patient is suffering from.

40. The method of claim 39 additionally comprising placing a fourth suture in the palpable regional lymph node, said fourth suture being coated with anti-CD3 and anti-CD28 antibodies.

41. The method of claim 37 wherein the head and neck cancer is head and neck squamous cell carcinoma.

42. The method of claim 37 additionally comprising:
removing the palpable regional lymph node during tumor resection;
expanding the lymph-node lymphocytes in vitro; and
infusing the expanded lymphocytes into the patient.

43. A method of adoptive immunotherapy comprising:
placing a suture coated with one or more immuno-stimulating molecules into the lymph node of a patient;
removing the lymph node of the patient;
expanding the lymph-node lymphocytes in vitro; and
infusing the expanded lymphocytes into the patient.

44. The methods of claim 43 wherein the suture is coated with anti-CD3 and anti-CD28.

45. A method of expanding lymphocytes in vitro comprising contacting the lymphocytes with surgical suture coated with anti-CD3 and anti-CD28.

46. A method of treating cancer in a patient comprising:
surgically resecting the tumor; and
placing a suture coated with an immuno-modulating compound in the floor of the wound created by the resection.

47. The method of claim 46 wherein the suture is coated with anti-CD3 and anti-CD28 monoclonal antibodies.

48. The method of claim 46 additionally comprising placing a suture coated with anti-microbial compounds in the wound.
